(19) United States
(12) Patent Application Publication
(10) Pub. No.: US 2023/0416188 A1
(43) Pub. Date: Dec. 28, 2023

(54) NEW SYNTHESIS OF L-PHENYLALANINE AMIDE

(71) Applicant: DSM IP ASSETS B.V., Heerlen (NL)

(72) Inventors: Werner BONRATH, Kaiseraugst (CH); Alexander GAA, Kaiseraugst (CH); Ralph HAERTER, Kaiseraugst (CH)

(21) Appl. No.: 18/253,175

(22) PCT Filed: Nov. 9, 2021
(86) PCT No.: PCT/EP2021/081030

§ 371 Date: May 16, 2023

(30) Foreign Application Priority Data
Nov. 18, 2020 (EP) 20208400.0

(51) Int. Cl. C07C 231/02

(52) U.S. Cl. CPC C07C 231/02; C07B 2200/07

(57) ABSTRACT

The present invention relates to a process for producing L-phenylalanine amide.

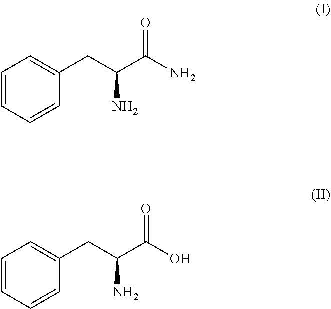 [SMILES: NC(=O)[C@@H](N)Cc1ccccc1 ; N[C@@H](Cc1ccccc1)C(=O)O]

The present invention relates to a process for producing L-phenylalanine amide.

L-Phenylalanine amide is an important amino acid amide, which can be used as such or which can be used as an intermediate in organic synthesis.

In U.S. Pat. No. 4,017,513, L-phenylalanine amide is produced in reaction mixture using benzene. This is crucial to obtained highly pure products.

Because L-phenylalanine amide is an important compound there is always a need for improved ways to produce it.

Surprisingly, it was found that when at least one alcohol is used as a solvent, the reaction results in excellent yield and the work-up is easy.

L-Phenylalanine amide is the following compound of formula (I)

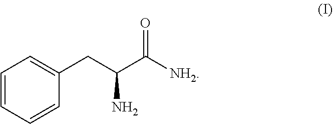 [SMILES: NC(=O)[C@@H](N)Cc1ccccc1]

The new and improved synthesis of L-phenylalanine amide allows to obtain L-phenylalanine amide in an excellent yield. Due the choice of alcohols instead of benzene the process of the present invention is much easier to work up.

Therefore the present invention relates to process (P) to produce L-phenylalanine amide, which is the compound of formula (I)

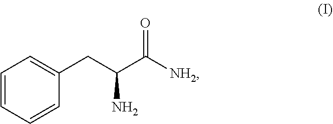 [SMILES: NC(=O)[C@@H](N)Cc1ccccc1]

wherein a first step (step (i) the compound of formula (II)

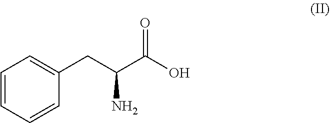 [SMILES: N[C@@H](Cc1ccccc1)C(=O)O]

is reacted with a solid acidic cation exchanger in the presence of at least one alcohol, and in a second step (step (ii)) the reaction mixture of step (i) is reacted with ammonia under pressure of at least 1.5 bar.

The process according to the present invention is usually carried out as following:

In a first step (step (i)) the compound of formula (II) is reacted with a solid acidic cation exchanger in the presence of the alcohol (or mixture of alcohols) at elevated temperatures.

Afterwards, in a second step (step (ii)) the reaction mixture of the first step is reacted with ammonia under pressure.

Finally the product (compound of formula (I)) is removed from the reaction mixture and purified.

The reaction scheme of the process according to the present invention looks like following (when the alcohol is ethanol):

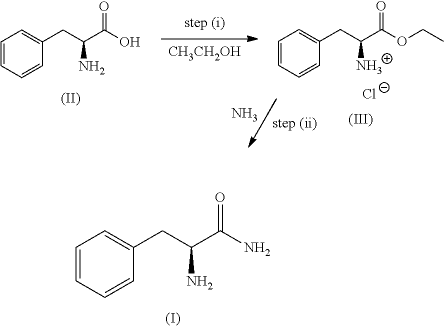

As stated above the process according to the present invention is carried out in the presence of at least one alcohol. The alcohol is used in an excess in view of the compound of formula (II). The at least one alcohol serves as solvent as well. Therefore no further solvent (other than the at least one alcohol) is needed and used.

Suitable alcohols according to the invention are any primary, secondary and tertiary alcohols, which are commonly used as solvents.

Preferred alcohols are aliphatic alcohols, which can be primary, secondary or tertiary ones.

More preferred are aliphatic alcohols with a C₁-C₆-alkyl moiety, which can be linear or branched and which can be primary, secondary or tertiary alcohols.

Most preferred are alcohols such as methanol, ethanol, propanol, butanol, pentanol, hexanol, sec-hexanol, sec-butanol and tert-butanol. Especially preferred is methanol and/or ethanol.

The at least one alcohol is used in an excess in view of the compound of formula (II). The molar ratio of the alcohol to the compound of formula (II) is usually at least 2:1. The upper limit is not essential for the invention. Usually it is up to 100:1. A preferred molar ratio of the alcohol to the compound of formula (II) is usually at least 10:1 to 50:1.

Therefore the present invention relates to process (P1), which is process (P), wherein the alcohol (or mixture of alcohols) are primary, secondary or tertiary aliphatic alcohols.

Therefore the present invention relates to process (P1’), which is process (P), wherein the at least one alcohol is an aliphatic alcohol with a C₁-C₆-alkyl moiety, which can be linear or branched and which can be primary, secondary or tertiary alcohols.

Therefore the present invention relates to process (P1”), which is process (P), wherein the at least one alcohol is chosen from the group consisting of methanol, ethanol, propanol, butanol, pentanol, hexanol, sec-hexanol, sec-butanol and tert-butanol.

Therefore the present invention relates to process (P1’”), which is process (P), wherein the at least one alcohol is chosen from the group consisting of methanol and ethanol.

Therefore the present invention relates to process (P2), which is process (P), (P1), (P1’), (P1”) or (P1’”), wherein the molar ratio of the alcohol to the compound of formula (II) is at least 2:1.

Therefore the present invention relates to process (P2’), which is process (P), (P1), (P1’), (P1”) or (P1’”), wherein the molar ratio of the alcohol to the compound of formula (II) is 2:1 to 100:1.

Therefore the present invention relates to process (P2”), which is process (P), (P1), (P1’), (P1”) or (P1’”), wherein the molar ratio of the alcohol to the compound of formula (II) is 10:1 to 50:1.

Therefore the present invention relates to process (P3), which is process (P), (P1), (P1’), (P1”), (P1’”), (P2), (P2’) or (P2”), wherein step (i) no further solvent (next to the at least one alcohol) is used.

The step (i) of the process according to the present invention is carried out in the presence of a solid acidic cation exchanger. This solid acidic cation exchanger can be any commonly used solid acidic cation exchanger. Usually they are of a resin base. Such solid acidic cation exchanger can be bought commercially and are known under tradenames such as i.e. Dowex® and Amberlite®.

Usually step (i) is carried out at an elevated temperature. A suitable temperature range for this step of the process according to the present invention goes from 30° C.-150° C. (preferably from 40° C.-130° C., more preferably 50° C.-120° C.).

Therefore the present invention relates to process (P4), which is process (P), (P1), (P1’), (P1”), (P1’”), (P2), (P2’), (P2”) or (P3), wherein step (i) is carried out at an elevated temperature.

Therefore the present invention relates to process (P4’), which is process (P4), wherein the temperature goes from 30° C.-150° C.

Therefore the present invention relates to process (P4”), which is process (P4), wherein the temperature goes from 40° C.-130° C.

Therefore the present invention relates to process (P4’”), which is process (P4), wherein the temperature goes from 50° C.-120° C.

Usually step (i) of the process according to the present invention is carried out at ambient pressure.

Therefore the present invention relates to process (P5), which is process (P), (P1), (P1’), (P1”), (P1’”), (P2), (P2’), (P2”), (P3), (P4), (P4’), (P4”) or (P4’”), wherein step (i) is carried out at an ambient pressure.

Afterwards when the reaction of step (i) has been carried out, ammonia (in gas form) is added to the reaction mixture (as obtained from step (i)) and pressure is applied. No further solvent is added at step (ii). This is due to fact that in step (i) the at least alcohol is added in excess in view of the compound of formula (II).

Ammonia is usually added in excess (in regard to the starting material, which is the compound of formula (II)).

The pressure which is applied in step (ii) is at least 1.5 bar. Usually at a pressure of 1.5-20 bar is applied. Preferably a pressure of 3 to 15 bar is applied.

Therefore the present invention relates to process (P6), which is process (P), (P1), (P1’), (P1”), (P1’”), (P2), (P2’), (P2”), (P3), (P4), (P4’), (P4”), (P4’”) or (P5), wherein step (ii) is carried out at a pressure of at least 1.5 bar.

Therefore the present invention relates to process (P6’), which is process (P6), wherein step (ii) is carried out at a pressure of 1.5-20 bar.

Therefore the present invention relates to process (P6”), which is process (P6), wherein step (ii) is carried out at a pressure of 3-15 bar.

Therefore the present invention relates to process (P7), which is process (P), (P1), (P1’), (P1”), (P1’”), (P2), (P2’), (P2”), (P3), (P4), (P4’), (P4”), (P4’”), (P5), (P6), (P6’) or (P6’”), wherein step (ii) no further solvent is added.

Usually step (ii) is carried out at room temperature (18-25° C.) This means that the reaction mixture of step is not heated.

Therefore the present invention relates to process (P8), which is process (P), (P1), (P1’), (P1”), (P1’”), (P2), (P2’), (P2”), (P3), (P4), (P4’), (P4”), (P4’”), (P5), (P6), (P6’), (P6”) or (P7), wherein step (ii) is carried out at room temperature (18-25° C.).

Afterwards the product (compound of formula (I)) is isolated from the reaction mixture (and optionally purified) by usual means.

L-Phenylalanine amide (compound of formula (I)) is obtained in excellent yields.

The following Example illustrates the invention further without limiting it. All percentages and parts, which are given, are related to the weight and the temperatures are given in ° C., when not otherwise stated.

### EXAMPLES

### Example 1

In a 350-ml four-necked flask equipped with a KPG-stirrer, thermometer, and a reflux condenser with an argon inlet was charged with 10.2 g (60.5 mmol ) L-phenylalanine, 13.2 g Dowex 50WX8 hydrogen form (dried) and 120 ml ethanol (2055 mmol). The mixture was stirred at 400 rpm and heated at reflux 78° C. (100° C. oil) for 16 h. The mixture was cooled to room temperature and transferred in a 500 ml autoclave filled with 96 g ammonia. The mixture was stirred at 20° C. at 1000 rpm at 6 bar for 4 days. The reaction mixture was sucked out and the Dowex was filtered and washed with ethanol and toluene. The filtrate was evaporated under reduced pressure (10 mbar, 40° C.). 10.01 g L-phenylalanine-amide with a yield of 65% with a purity of 64.5% is obtained

1. Process to produce L- the compound of formula (I) 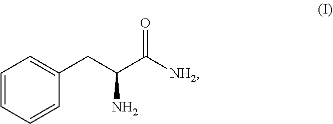 [SMILES: NC(=O)[C@@H](N)Cc1ccccc1]
wherein a first step (step (i) the compound of formula (II) 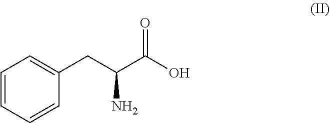 [SMILES: N[C@@H](Cc1ccccc1)C(=O)O]
is reacted with a solid acidic cation exchanger in the presence of at least one alcohol, and in a second step (step (ii)) the reaction mixture of step (i) is reacted with ammonia under pressure of at least 1.5 bar.

2. Process according to claim 1, wherein the at least one alcohol is an aliphatic alcohol with a C₁-C₆-alkyl moiety, which can be linear or branched and which can be primary, secondary or tertiary alcohols.

3. Process according to claim 1, wherein the at least one alcohol is chosen from the group consisting of methanol, ethanol, propanol, butanol, pentanol, hexanol, sec-hexanol, sec-butanol and tert-butanol.

4. Process according to claim 1, wherein the molar ratio of the alcohol to the compound of formula (II) is at least 2:1.

5. Process according to claim 1, wherein step (i) is carried out at a temperature of from 30° C.-150° C.

6. Process according to claim 1, wherein step (i) is carried out at an ambient pressure.

7. Process according to claim 1, wherein step (ii) is carried out at a pressure of at least 1.5 bar.

8. Process according to claim 7, wherein the pressure is 1.5-20 bar.

9. Process according to claim 1, wherein step (ii) no further solvent is added to the reaction mixture.

10. Process according to claim 1, wherein step (ii) is carried out at room temperature (18-25° C.).
